(19) United States
(12) Patent Application Publication
(10) Pub. No.: US 2024/0319175 A1
(43) Pub. Date: Sep. 26, 2024

(54) Fetal Microchimeric Cells and EVs for Regenerative Medicine in Women's Health

(71) Applicants: Board of Regents, The University of Texas System, Austin, TX (US); University of Vermont and State Agricultural College, Burlington, VT (US)

(72) Inventors: Ramkumar Menon, Galveston, TX (US); Ananth Kumar Kammala, Galveston, TX (US); Elizabeth Bonney, Galveston, TX (US)

(21) Appl. No.: 18/614,071

(22) Filed: Mar. 22, 2024

Related U.S. Application Data
(60) Provisional application No. 63/491,676, filed on Mar. 22, 2023.

(51) Int. Cl. G01N 33/50; A61K 35/12; G16H 50/30

(52) U.S. Cl. CPC G01N 33/5091; A61K 35/12; G16H 50/30; G01N 2800/50

(57) ABSTRACT

Provided herein are methods and compositions for determining an increased risk of maternal cardiovascular disease caused by an infection comprising: obtaining, or having obtained, a biological sample from a subject at risk of, of having had a pre-term birth; measuring fetal microchimeric cells or fetal extracellular vesicles in the biological sample; and using a machine learning algorithm calculating a risk of cardiovascular disease based on the an increase or a decrease in fetal microchimeric cells in the biological sample when compared to a maternal sample from a subject that does not have an infection.

### CROSS-REFERENCE TO RELATED APPLICATIONS

This application claims priority to U.S. Provisional Application Ser. No. 63/491,676, filed Mar. 22, 2023, the entire contents of which are incorporated herein by reference.

### STATEMENT OF FEDERALLY FUNDED RESEARCH

This invention was made with government support under awarded by the National Institutes of Health/NSF/DARPA. The government has certain rights in the invention.

### TECHNICAL FIELD OF THE INVENTION

The present invention relates in general to the field of fetal microchimeric cells and/or fetal extracellular vesicles (EVs) regenerative medicine in women's health.

### INCORPORATION-BY-REFERENCE OF MATERIALS FILED ON COMPACT DISC

Not applicable.

### BACKGROUND OF THE INVENTION

Without limiting the scope of the invention, its background is described in connection with heart disease.

In the US, heart disease kills a woman every minute¹. Heart disease is different in women and men²⁻⁴. However, while the female hormonal milieu may contribute to sex-specific cardiovascular physiology, focus on these complexities^{5,6} has yet to provide a successful intervention. Thus, understanding other mitigating factors is critical for women's overall health. Sex influences vascular disease systemically⁷⁻⁹ and in specific vascular beds^{10 11}. Sex differences in platelet function¹², sympathetic nerve activityl³, vascular responsiveness¹⁴, the composition of perivascular adipose tissue¹⁵, and other basic metabolic parameters¹⁶ all feed into the sex differences observed in disease risk. Hypertension, diabetes and metabolic syndrome, stress, and smoking are stronger risk factors for heart disease in women than in men¹⁷. All of these are modified by pregnancy. Thus, pregnancy is a critical determinant of sex-specific cardiovascular risk. In women, ischemic heart disease and cardiomyopathy with preserved ejection fraction and heart failure are common, creating a significant disease burden¹⁸. Such cardiomyopathy in older women is related to cellular senescence of endothelial cells, valvular interstitial cells, cardiomyocytes, etc., intriguingly via very similar molecular pathways (senescence) observed in fetal tissues (fetal membranes and placenta) in human parturition¹⁹⁻²⁶. This contrasts with dilated cardiomyopathy seen in men or in peripartum women^{27,28}.

Pregnancy is a window for future health for the fetus and the mother. Spontaneous preterm birth (PTB) is a major public health concern, impacting 15 million pregnancies and 1 million neonatal deaths/per year globally²⁹. The impact of PTB on subsequent maternal cardiovascular health is also significant. This proposal will provide a viable mechanism linking fetal cell trafficking in PTB to subsequent maternal heart disease. Importantly, it proposes to mechanistically explain the observed association of increased cardiovascular disease risk with decreased gestational age at delivery.

### SUMMARY OF THE INVENTION

As embodied and broadly described herein, an aspect of the present disclosure relates to a method of determining an increased risk of maternal cardiovascular disease caused by an infection comprising: obtaining, or having obtained, a biological sample from a subject at risk of, of having had a pre-term birth; measuring fetal microchimeric cells or fetal extracellular vesicles in the biological sample; and using a machine learning algorithm calculating a risk of cardiovascular disease based on an increase or a decrease in fetal microchimeric cells or fetal extracellular vesicles in the biological sample when compared to a maternal sample from a subject that does not have an infection. In one aspect, there is an increase in a frequency of fetal microchimeric cells or fetal extracellular vesicles expressed high CD14 or CD14low, CD11c, CD86 and IFN-γ which is indicative of a subpopulation of CD11c+ M1 macrophages or dendritic cell phenotypes. In another aspect, there is an increase in a frequency of fetal microchimeric cells or fetal extracellular vesicles expressed CD117 (c-kit) and Ly6A/E (Sca-1) and showed negative expression for lineage markers CD4, CD8, and CD11b (Mac-1), consistent with the Lineage-Sca-1+c-kit+ (LSK+) phenotype of murine hematopoictic cells. In another aspect, there is an increase in a frequency of fetal microchimeric cells or fetal extracellular vesicles comprising Lineage-Sca-1+c-kit− (LSK−) phenotype of murine hematopoietic cells called very small embryonic-like (VSEL) cells. In another aspect, there is a decrease in a frequency of fetal microchimeric cells or fetal extracellular vesicles the monocyte-macrophage marker CD14, the myeloid marker CD11b, and F4/80, while being negative for macrophage polarization markers, indicating a non-activated macrophage phenotype. In another aspect, there is a decrease in a frequency of fetal microchimeric cells or fetal extracellular vesicles expressed both CD4 and CD8, and low T cell receptor, characteristic of double-positive thymocytes. In another aspect, there is a decrease in a frequency of fetal microchimeric cells or fetal extracellular vesicles of non-hematopoietic that do not express CD45. In another aspect, the subject also has an increase in a risk of hypertension, coronary artery calcification, type 2 diabetes mellitus and hypercholesterolemia. In another aspect, the biological sample is a blood, a plasma, cardiac, a kidney, or a lung tissue. In another aspect, based on the calculated risk of cardiovascular disease is an increase in a risk of cardiovascular disease as a result of an infection administering one or more anti-infective agent, antibiotic agent, or antimicrobial agent to the subject to treat the infection. In another aspect, the antibiotic is selected from the group consisting of: azithromycin penicillins, cephalosporines, tetracyclines, sulphonamides, aminoglycosides, aminocyclitols, macrolides, quinolones, ionophores, carbadox, nitrofuran antibiotics, phenicols, a mixture thereof, and any combination thereof. In another aspect, the antibiotic is selected from the group consisting of: a macrolide, aminoglycoside, polymyxin, a tetracycline, a cephalosporin, a quinolone or a fluoroquinolone; amikacin, apramycin, gentamicin, kanamycin, neomycin, tobramycin, paromomycin, streptomycin, spectinomycin, plazomicin, ertapenem, doripenem, imipenem/cilastatin, meropenem, cefadroxil, cefazolin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, clindamycin, lincomycin, lipopeptide, daptomycin, azithromycin, clarithromycin, dirithromycin, erythromycin, roxithromycin, troleandomycin, telithromycin, spiramycin, fidaxomicin, tulathromycin, aztreonam, linezolid, posizolid, radezolid, torezolid, amoxicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, bacitracin, colistin, polymyxin b, besifloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, sulfonamidochrysoidine, demeclocycline, doxycycline, minocycline, oxytetracycline, tetracycline, arsphenamine, chloramphenicol, fosfomycin, fusidic acid, metronidazole, mupirocin, platensimycin, quinupristin/dalfopristin, thiamphenicol, tigecycline, tinidazole, trimethoprim, clofazimine, dapsone, capreomycin, cycloserine, ethambutol, ethionamide, isoniazid, pyrazinamide, rifampicin, rifabutin, or rifapentine, or a pharmaceutically acceptable salt thereof. In another aspect, the machine learning algorithm is selected from perplexity, learning rate (eta), K-Nearest Neighbors algorithm (exact, vantage point tree), and gradient algorithm (Barnes-Hut) and phenograph K-nearest-neighbor density-based clustering algorithm.

As embodied and broadly described herein, an aspect of the present disclosure relates to method of treating a subject that is pregnant and suspected of having an increased risk of maternal cardiovascular disease caused by an infection comprising: obtaining, or having obtained, a biological sample from a subject at risk of, of having had a pre-term birth; measuring fetal microchimeric cells or fetal extracellular vesicles in the biological sample; and using a machine learning algorithm calculating a risk of cardiovascular disease based on an increase or a decrease in fetal microchimeric cells or fetal extracellular vesicles in the biological sample when compared to a maternal sample from a subject that does not have an infection; and based on the increased risk of cardiovascular disease administering one or more anti-infective agents, antibiotic agents, or antimicrobial agents to the subject. In one aspect, the method detects at least one of: an increase in a frequency of fetal microchimeric cells or fetal extracellular vesicles expressed high CD14 or CD14low, CD11c, CD86 and IFN-γ which is indicative of a subpopulation of CD11c+ M1 macrophages or dendritic cell phenotypes; an increase in a frequency of fetal microchimeric cells or fetal extracellular vesicles expressed CD117 (c-kit) and Ly6A/E (Sca-1) and showed negative expression for lineage markers CD4, CD8, and CD11b (Mac-1), consistent with the Lineage-Sca-1+c-kit+ (LSK+) phenotype of murine hematopoietic cells; or an increase in a frequency of fetal microchimeric cells or fetal extracellular vesicles comprising Lineage-Sca-1+c-kit− (LSK−) phenotype of murine hematopoietic cells called very small embryonic-like (VSEL) cells. In another aspect, method detects at least one of: a decrease in a frequency of fetal microchimeric cells or fetal extracellular vesicles the monocyte-macrophage marker CD14, the myeloid marker CD11b, and F4/80, while being negative for macrophage polarization markers, indicating a non-activated macrophage phenotype; a decrease in a frequency of fetal microchimeric cells or fetal extracellular vesicles expressed both CD4 and CD8, and low T cell receptor, characteristic of double-positive thymocytes; or a decrease in a frequency of fetal microchimeric cells or fetal extracellular vesicles of non-hematopoietic that do not express CD45. In another aspect, the subject also has an increase in a risk of hypertension, coronary artery calcification, type 2 diabetes mellitus and hypercholesterolemia. In another aspect, the biological sample is a blood, a plasma, cardiac, a kidney or a lung tissue. In another aspect, the antibiotic is selected from the group consisting of: azithromycin penicillins, cephalosporines, tetracyclines, sulphonamides, aminoglycosides, aminocyclitols, macrolides, quinolones, ionophores, carbadox, nitrofuran antibiotics, phenicols, a mixture thereof, and any combination thereof. In another aspect, the antibiotic is selected from the group consisting of: a macrolide, aminoglycoside, polymyxin, a tetracycline, a cephalosporin, a quinolone or a fluoroquinolone; amikacin, apramycin, gentamicin, kanamycin, neomycin, tobramycin, paromomycin, streptomycin, spectinomycin, plazomicin, ertapenem, doripenem, imipenem/cilastatin, meropenem, cefadroxil, cefazolin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, clindamycin, lincomycin, lipopeptide, daptomycin, azithromycin, clarithromycin, dirithromycin, erythromycin, roxithromycin, troleandomycin, telithromycin, spiramycin, fidaxomicin, tulathromycin, aztreonam, linezolid, posizolid, radezolid, torezolid, amoxicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, bacitracin, colistin, polymyxin b, besifloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, sulfonamidochrysoidine, demeclocycline, doxycycline, minocycline, oxytetracycline, tetracycline, arsphenamine, chloramphenicol, fosfomycin, fusidic acid, metronidazole, mupirocin, platensimycin, quinupristin/dalfopristin, thiamphenicol, tigecycline, tinidazole, trimethoprim, clofazimine, dapsone, capreomycin, cycloserine, ethambutol, ethionamide, isoniazid, pyrazinamide, rifampicin, rifabutin, or rifapentine, or a pharmaceutically acceptable salt thereof. In another aspect, the machine learning algorithm is selected from perplexity, learning rate (cta), K-Nearest Neighbors algorithm (exact, vantage point tree), and gradient algorithm (Barnes-Hut) and phenograph K-nearest-neighbor density-based clustering algorithm.

As embodied and broadly described herein, an aspect of the present disclosure relates to a computerized method for determining an increased risk of maternal cardiovascular disease caused by an infection comprising: obtaining, or having obtained, a biological sample from a subject at risk of, of having had a pre-term birth; measuring fetal microchimeric cells or fetal extracellular vesicles in the biological sample; and using a processor with a non-transitory computer readable medium and a machine learning algorithm to calculate a risk of cardiovascular disease based on an increase or a decrease in fetal microchimeric cells or fetal extracellular vesicles in the biological sample when compared to a maternal sample from a subject that does not have an infection.

As embodied and broadly described herein, an aspect of the present disclosure relates a method of treating a subject in need of regenerating or protecting one or more maternal organs comprising: isolating at least one of: fetal microchimeric cells or fetal extracellular vesicles comprising stem cell-like properties from a pregnant female during pregnancy; and injecting the fetal microchimeric cells or fetal extracellular vesicles into a patient in need thereof in an amount sufficient to regenerate and protect the one or more maternal organs. In one aspect, the one or more organs are selected from heart, kidney, or lung. In another aspect, the cells are not obtained from a pregnant female subject with a premature birth or preterm pregnancy. In another aspect, the at least one of fetal microchimeric cells or fetal extracellular vesicles are selected from: fetal microchimeric cells or fetal extracellular vesicles that express high CD14 or CD14low, CD11c, CD86 and IFN-γ which is indicative of a subpopulation of CD11c+ M1 macrophages or dendritic cell phenotypes; fetal microchimeric cells or fetal extracellular vesicles that express CD117 (c-kit) and Ly6A/E (Sca-1) and showed negative expression for lineage markers CD4, CD8, and CD11b (Mac-1), consistent with Lineage-Sca-1+c-kit+ (LSK+) phenotype of murine hematopoietic cells; or fetal microchimeric cells or fetal extracellular vesicles that express comprising Lineage-Sca-1+c-kit− (LSK−) phenotype of murine hematopoietic cells called very small embryonic-like (VSEL) cells; fetal microchimeric cells or fetal extracellular vesicles comprising monocyte-macrophage marker CD14, myeloid marker CD11b, and F4/80, while being negative for macrophage polarization markers, indicating a non-activated macrophage phenotype; fetal microchimeric cells or fetal extracellular vesicles comprising that express both CD4 and CD8, and low T cell receptor, characteristic of double-positive thymocytes; or fetal microchimeric cells or fetal extracellular vesicles of non-hematopoietic that do not express CD45.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C show the methodology framework for high-dimensional mass cytometry. (FIG. 1A) Male homozygous mT/mG+/+ C57BL/6J mice were mated with wild-type females, which resulted in progeny cells that express mT+. Pregnant mice were treated intravaginally with 104 CFU E. coli to mimic ascending infection, with sterile nutrient broth serving as negative control. (FIG. 1B) Both flow cytometry and mass cytometry utilize cell labeling with antibodies to facilitate single cell analysis, although fewer spectral overlap is observed with mass cytometry. (FIG. 1C) Molecules of interest were tagged with elemental isotope labelled antibodies. Following nebulization and injection into a mass analyzer, the ionized metals were accelerated through a vacuum chamber and were analyzed based on time of flight.

FIG. 2 shows fluorescence imaging of fetal and maternal tissues at E16. Sections of placenta, maternal uterine and maternal heart tissues at E16 were viewed under both Texas Red and DAPI filters to visualize mT+ and nuclear stain, respectively. Inset shows increased magnification of overlaid maternal heart tissue image. Negative control used was maternal heart from wild-type female C57BL/6J mated with wild-type male C57BL/6J. Scale bar: 50 μm.

FIGS. 3A to 3D show phenograph cluster profiles of cells found in maternal heart at E16 during normal pregnancy and ascending infection. (FIG. 3A) Heatmap of 38 clusters generated by phenograph, the various cell markers present, and their frequency in both ascending infection and normal pregnancy. Columns with black labels show the expression levels of a certain cell marker in each cluster, with yellow as the maximum marker intensity and blue as the minimum marker intensity. Columns with red labels at the rightmost side portray the frequency of each cluster in ascending infection and normal pregnancy, with gold as the highest frequency and black as the lowest frequency. (FIG. 3B) Location of clusters 1, 3, 5, 6, 7, and 8 in the t-SNE plot. (FIG. 3C) Cell clusters present in ascending infection superimposed on t-SNE plot. (FIG. 3D) Cell clusters present in normal pregnancy superimposed on t-SNE plot.

FIGS. 4A to 4D show phenograph cluster profiles of cells found in maternal heart at postpartum day 28 following normal pregnancy and ascending infection. (FIG. 4A) Heatmap of 34 clusters generated by phenograph, the various cell markers present, and their frequency in both ascending infection and normal pregnancy. Columns with black labels show the expression levels of a certain cell marker in each cluster, with yellow as the maximum marker intensity and blue as the minimum marker intensity. Columns with red labels at the rightmost side portray the frequency of each cluster in ascending infection and normal pregnancy, with gold as the highest frequency and black as the lowest frequency. (FIG. 4B) Location of clusters 1, 3, 4, 5, 7, 8, and 9 in the t-SNE plot. (FIG. 4C) Cell clusters present in ascending infection superimposed on t-SNE plot. (FIG. 4D) Cell clusters present in normal pregnancy superimposed on t-SNE plot.

FIGS. 5A to 5C show Postpartum cardiac physiologic parameters following normal pregnancy and ascending infection. (FIG. 5A) Echocardiographic measurements to determine cardiac output and systolic ejection fraction. (FIG. 5B) Postpartum cardiac output in normal pregnancy and ascending infection, with equivalent measurements for nonpregnant mice. (FIG. 5C) Postpartum systolic ejection fraction in normal pregnancy and ascending infection, with equivalent measurements for nonpregnant mice.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention includes methods and compositions for determining an increased risk of maternal cardiovascular disease caused by an infection comprising: obtaining, or having obtained, a biological sample from a subject at risk of, of having had a pre-term birth; measuring fetal microchimeric cells or fetal extracellular vesicles in the biological sample; and using a machine learning algorithm calculating a risk of cardiovascular disease based on an increase or a decrease in fetal microchimeric cells and fetal extracellular vesicles in the biological sample when compared to a maternal sample from a subject that does not have an infection.

The present invention also includes fetal microchimeric cells and/or fetal extracellular vesicles coming from a mother during pregnancy can help her to regenerate tissues, for example, syngencic or allogeneic grafts. These tissues ending up in heart and lung have stem cell-like properties that can regenerate and protect maternal organs. If these cells come from premature birth (preterm pregnancies), they are different type and do not have the same number or characteristics of a stem cell capable of repairing the maternal organs. Thus, the fetal microchimeric cells and fetal extracellular vesicles can be isolated based on the teachings herein and used for regenerative purposes of organs and tissues in mother. The fetal microchimeric cells and/or fetal extracellular vesicles are isolated as described herein and one or more of the cell populations, e.g., fetal microchimeric cells or fetal extracellular vesicles selected from at least one of: fetal microchimeric cells or fetal extracellular vesicles that express high CD14 or CD14low, CD11c, CD86 and IFN-γ which is indicative of a subpopulation of CD11c+ M1 macrophages or dendritic cell phenotypes; fetal microchimeric cells or fetal extracellular vesicles that express CD117 (c-kit) and Ly6A/E (Sca-1) and showed negative expression for lineage markers CD4, CD8, and CD11b (Mac-1), consistent with Lineage-Sca-1+c-kit+ (LSK+) phenotype of murine hematopoictic cells; or fetal microchimeric cells or fetal extracellular vesicles that express comprising Lineage-Sca-1+c-kit− (LSK−) phenotype of murine hematopoietic cells called very small embryonic-like (VSEL) cells; fetal microchimeric cells or fetal extracellular vesicles comprising monocyte-macrophage marker CD14, myeloid marker CD11b, and F4/80, while being negative for macrophage polarization markers, indicating a non-activated macrophage phenotype; fetal microchimeric cells or fetal extracellular vesicles comprising that express both CD4 and CD8, and low T cell receptor, characteristic of double-positive thymocytes; or fetal microchimeric cells or fetal extracellular vesicles of non-hematopoietic that do not express CD45, and provided to the recipient in an amount sufficient to prevent or trigger tissue regeneration.

To achieve the present invention used a Cre reporter mouse model system, which when mated with wild-type C57BL/6J females resulted in cells and tissues of progeny expressing red fluorescent protein tandem dimer Tomato (mT+), was used to detect fetal microchimeric cells. On embryonic day (E)15, 10⁴ colony-forming units (CFU) E. coli was administered intravaginally to mimic ascending infection, with delivery on or before E18.5 considered as preterm delivery. A subset of pregnant mice was sacrificed at E16 and postpartum day 28 to harvest maternal hearts. Heart tissues were processed for immunofluorescence microscopy and high-dimensional mass cytometry by time-of-flight (CyTOF) using an antibody panel of immune cell markers. Changes in cardiac physiologic parameters were measured up to 60 days postpartum via two-dimensional echocardiography.

It was found that intravaginal E. coli administration resulted in preterm delivery of live pups in 70% of the cases. mT+ expressing cells were detected in maternal uterus and heart, implying that fetal cells can migrate to different maternal compartments. During ascending infection, more fetal antigen-presenting cells (APCs) and less fetal hematopoietic stem cells (HSCs) and fetal double-positive (DP) thymocytes were observed in maternal hearts at E16 compared to normal pregnancy. These HSCs were cleared while DP thymocytes persisted 28 days postpartum following an ascending infection. No significant changes in cardiac physiologic parameters were observed postpartum except a trend in lowering the ejection fraction rate in preterm delivered mothers.

Thus, during both normal pregnancy and ascending infection distinct subpopulations of fetal microchimeric immune cells were found within the maternal heart, which influence the maternal cardiac microenvironment via (1) modulation of cardiac reverse modeling processes by fetal stem cells, and (2) differential responses to recognition of fetal APCs by maternal T cells. An increase in certain subpopulations were indicative of increased risk, which a decrease in other subpopulations also led to an increase risk of infection and negative effects in cardiac, lung, and kidney tissues.

Fetal microchimerism refers to the presence of a small number of fetal cells in the maternal body (1). During pregnancy, fetal cells such as fetal stem cells or immune cells can cross the placenta and enter the maternal circulation. These fetal cells can then migrate and integrate into various maternal tissues and organs (2). As a result, the mother may retain a small population of cells that originated from her fetus. These cells can persist in the maternal body for years or even decades after pregnancy (3, 4), but may also be impacted by the maternal immune system (5, 6). The relationship between maternal health outcomes and fetal microchimerism is an area of active research and ongoing investigation.

While the precise mechanisms and implications are not yet fully understood, some studies have suggested potential associations between fetal microchimerism and certain maternal health conditions. Fetal cells that persist in maternal tissues have been proposed to possess regenerative properties and have the potential to differentiate into various cell types, thus contributing to tissue repair and regeneration (7, 8). They were also implicated in the development or modulation of autoimmune diseases such as systemic sclerosis (9), rheumatoid arthritis (10), and systemic lupus erythematosus (10, 11). The characteristics of fetal microchimeric cells in women with normal pregnancy and pregnancies complicated by preterm birth (delivery of an offspring before 37 weeks of gestation), specifically spontaneous preterm births of unknown etiologies, are expected to differ.

Spontaneous preterm birth, predominantly associated with ascending infection, results in significant perinatal morbidity and mortality (12, 13) and can lead to long-term neurodevelopmental impairment (14) and chronic health problems to the baby (15). Preterm delivery also presents substantial risk to the mother for immediate complications such as hemorrhage, infection and admission into intensive care unit (16), postnatal depression (17), and chronic conditions such as hypertension, type 2 diabetes mellitus, and hypercholesterolemia (18). Women who delivered preterm are also at higher risk for ischemic heart disease with an adjusted hazard ratio of 2.47 (95% CI, 2.16-2.82) 10 years following delivery, with further increases in risk with every additional preterm delivery (19). Although an association was observed between high-risk blood pressure pattern in women who delivered preterm and coronary artery calcification, mechanisms linking preterm delivery and future maternal cardiovascular diseases are not fully understood (20).

Due to their ability to persist long-term and their differential responses (i.e., repair vs. autoimmune) depending on the context of pregnancy, the inventors hypothesize that fetal microchimeric cells can migrate to the maternal heart, and that their phenotype and functional potential may vary with normal and abnormal pregnancy, including preterm birth. Thus, the present inventors characterized fetal microchimeric immune cells in a cyclic recombinase (Cre)-reporter mouse model of ascending infection and preterm birth. By using high-dimensional mass cytometry by time-of-flight (CyTOF), different subpopulations of fetal microchimeric cells were identified in the maternal heart during pregnancy and after delivery. The studies herein serve as a crucial step in understanding function and potential impact of these cells on maternal cardiovascular health.

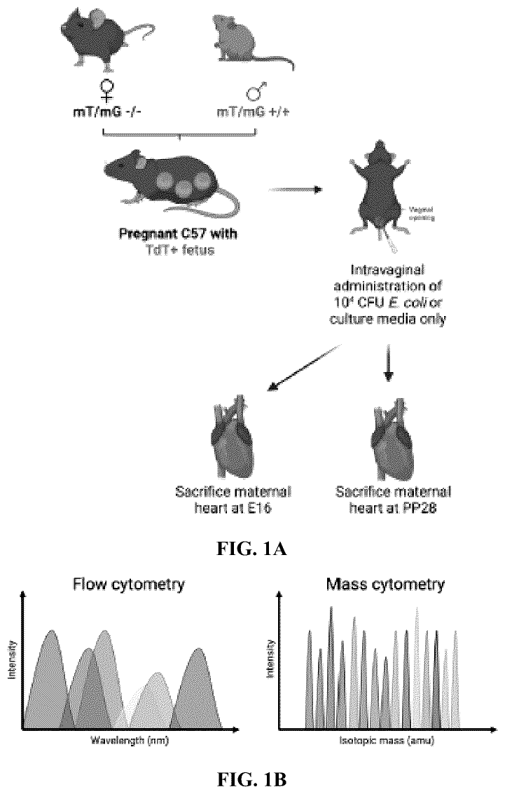

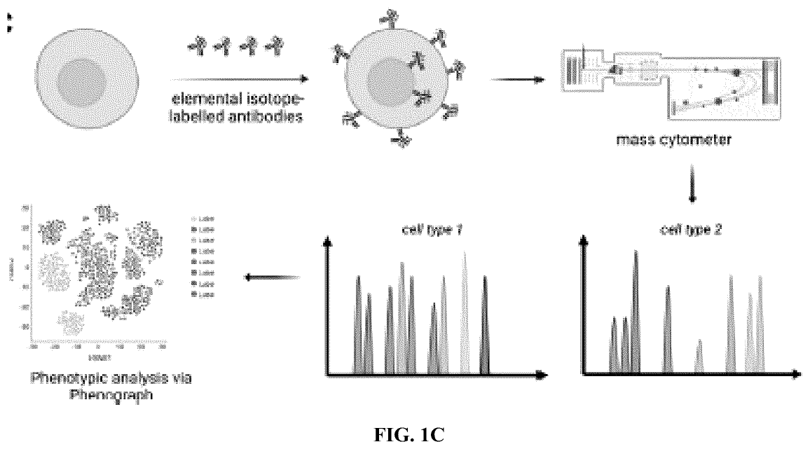

Animal care. A cyclic recombinase (Cre)-reporter mouse model was used to study fetal microchimeric cells as detailed by Sheller-Miller et al. (2019), wherein all cells and tissues of progeny expressed mT+ (21). Eight-to twelve-week old homozygous transgenic B6.129(Cg)-Gt(ROSA)26Sor^{tm4(ACTB-mT+,-EGFP)Luo/J} (mT/mG) males as previously described by Muzumdar et al. (22) (Strain #007676, The Jackson Laboratory, Bar Harbor, ME) were mated with wild type C57BL/6J females (Strain #000664, The Jackson Laboratory, Bar Harbor, ME) of the same age (FIG. 1A). The following morning, females observed with vaginal plug (indicating gestational day 0.5 (E0.5) were housed separately from the males. A cohort of nonpregnant mice were maintained for ultrasonographic studies. The mice were housed in a temperature- and humidity-controlled animal facility with 12-hour light and dark cycles, and regular chow and drinking solutions provided ad libitum. All animal procedures were approved by the Institutional Animal Care and Use Committee (IACUC) at the University of Texas Medical Branch at Galveston under protocol number 0411077E.

Escherichia coli (E. coli) culture. A day prior to experiment, a bacterial culture of ATCC 12014 Escherichia coli 055:K59(B5):H— (Lot #496291, ThermoFisher Scientific Remel Products, San Diego, CA, USA) was inoculated in 200 mL sterile nutrient broth (Difco, Cat. #234000, BD Biosciences, Franklin Lakes, NJ, USA) and cultured for 16 h at 37° C. with shaking. Using a spectrophotometer (D30 BioPhotometer, Eppendorf, Hamburg, Germany), OD₆₀₀ value was measured and colony forming units per milliliter (CFU/mL) was calculated based on the standard curve generated by Spencer et al. (2021) (23).

Model of ascending infection-induced preterm birth. At E15, pregnant mice were anesthetized via isoflurane inhalation. Based on CFU/mL calculation, 104 CFU of E. coli in 40 μL nutrient broth was administered vaginally to pregnant mice (n=13) using a blunted 200 μL pipette tip (FIG. 1A). The same volume of sterile nutrient broth was used in age-matched nonpregnant (n=3) and pregnant control groups (n=11). The timing of delivery was monitored using live cameras (Shenzen Wansview Technology, Shenzen, China). Delivery of the first pup on or before E18.5 was considered preterm. A subset of pregnant C57BL/6J mice (3 nutrient broth-treated, 5 E. coli-treated) were euthanized at E16 (1 day post-treatment) via carbon dioxide inhalation according to IACUC and the American Veterinary Medical Association guidelines, while the remaining mice (5 nutrient broth-treated, 5 E. coli-treated) were euthanized at postpartum day 28 (PP28).

Immunofluorescent imaging for mT+ signal in maternal heart tissues. Placental, and maternal uterine, heart, lung, kidney and brain specimens were collected after cuthanization. Visible placental fragments were gently removed, and the uterine lining was gently scraped to remove any adherent placental cells. The samples were then fixed in 4% paraformaldehyde overnight at 4° C. After fixation, the samples were washed twice with 1× phosphate-buffered saline (PBS) and subsequently placed in a 15% sucrose solution overnight at 4° C. They were then transferred to a 30% sucrose solution and stored at 4° C. until embedding in optimal cutting temperature (OCT) compound. Multiple 5 mm-sections were then incubated at room temperature for 30 minutes, followed by two washes in water to remove the OCT. The sections were incubated with DAPI for 2 minutes at room temperature, followed by two washes in water. To minimize autofluorescence, the tissues were treated with TrueVIEW Autofluorescence Quenching Kit (Vector Laboratories, Burlingame, CA) for 10 seconds, and washed twice with 1×Tris-buffered saline+Tween 20 (TBS-T). Subsequently, the slides were air dried at room temperature for 10 minutes and mounted using Mowiol 4-88 mounting medium.

Single-cell preparation from maternal heart tissues. To prepare the heart tissues, excess fat was carefully removed using fine forceps, and the tissues were washed with cold 1× phosphate-buffered saline (pH 7.4) and placed in RPMI 1640 with 10% fetal bovine serum and 1% penicillin-streptomycin. The heart tissues were then transferred to Accutase cell detachment solution (Corning, Corning, NY, USA) and cut into small pieces using fine scissors. After digestion for 60 min with gentle rocking at 37° C., the tissue samples were strained through a 70-μm cell strainer. The homogenates were washed twice with RPMI 1640 with 10% fetal bovine serum and 1% penicillin-streptomycin and centrifuged at 300 g for 5 min at 20° C. To remove erythrocytes, the cell pellets were resuspended in 1.0 mL red blood cell lysis buffer (BioLegend, San Diego, CA, USA), incubated for 10 minutes at room temperature, and diluted to 10 mL with RPMI 1640 with 10% fetal bovine serum and 1% penicillin-streptomycin. The samples were centrifuged at 300 g for 5 min at 20° C., and the resulting cell pellets were then resuspended in 0.5 mL commercial cell freezing media until use.

High-dimensional single-cell profiling of maternal heart tissues via mass cytometry by time-of-flight (CyTOF).

Antibodies. The basics of mass cytometry are summarized in FIGS. 1B and 1C. The antibodies used in this study were obtained either from the Flow Cytometry and Cellular Imaging Core Facility at The University of Texas MD Anderson Cancer Center in Houston, TX, USA, or custom-conjugated using the Maxpar antibody conjugation kit (Standard BioTools, Markham, ON, Canada) following the manufacturer's protocol. A comprehensive list of the antibodies utilized can be found in Supplementary Table 1. Following conjugation with their respective metal labels, the percentage yield was determined by measuring the absorbance at 280 nm using a Nanodrop 2000 spectrophotometer (Thermo Scientific, Wilmington, DE, USA). The antibodies were then diluted to a concentration of 0.3 mg/mL using Candor phosphate-buffered saline (PBS) antibody stabilization solution (Candor Bioscience GmbH, Wangen, Germany) and stored at 4° C. for future use.

Antibody staining. Single cells suspension samples were resuspended in Maxpar staining buffer for 10 minutes at room temperature on a shaker to block Fc receptors. Cells were mixed with a cocktail of metal-conjugated surface marker antibodies, yielding 500 μL final reaction volumes, and stained at room temperature for 30 min on a shaker. Following staining, cells were washed twice with Maxpar staining buffer. Next, cells were permeabilized with 4° C. Max Perm Buffer for 10 min at 4° C. Cells were then washed twice in Maxpar staining buffer to remove the remaining Max Perm. They were stained with intracellular antibodies in 500 μL for 30 min at room temperature on a shaker. Samples were then washed twice in Maxpar staining buffer. Cells were incubated overnight at 4° C. with 1 mL of 1:4,000 191/193Ir DNA intercalator (Standard BioTools, Markham, ON, Canada) diluted in Maxpar Fix/Perm overnight. The following day, cells were washed once with Maxpar staining buffer and then two times with deionized water (ddH₂O).

Mass cytometry. Prior to analysis, the cell pellet stained with antibodies and intercalated with DNA intercalator was resuspended in ddH₂O containing polystyrene normalization beads. The normalization beads contained lanthanum-139, prascodymium-141, terbium-159, thulium-169, and lutetium-175, following the method described by Finck et al. (2013) (24). Stained cells were then analyzed using a CyTOF 2 instrument (Standard BioTools Inc, Markham, ON, Canada) equipped with a Super Sampler sample introduction system (Victorian Airship & Scientific Apparatus, Alamo, CA, USA). The event rate was set to 200 to 300 cells per second. To ensure accurate data normalization, all mass cytometry files were normalized using the mass cytometry data normalization algorithm premessa available at github.com/ParkerICI/premessa/.

Data analysis. The Flow cytometry standard (.fcs) files were analyzed using FlowJo v10.9.0 (FlowJo LLC, Ashland, OR, USA). To ensure data quality, intact live single cells were manually gated using the Standard BioTools (Markham, ON, Canada) clean-up procedure. Each sample was assigned a unique Sample ID, and all samples were combined into a single .fcs file for further analysis. The concatenated file was subjected to 1-distributed stochastic neighbor embedding (t-SNE) in FlowJo, utilizing equal numbers of cells from both the normal pregnancy and ascending infection mouse groups, along with all phenotypic markers. The following settings were used: iterations (3000), perplexity (30), learning rate (cta) (8960), KNN algorithm (exact, vantage point trec), and gradient algorithm (Barnes-Hut). To explore the phenotypic diversity of immune cell populations in the maternal hearts of different mouse groups, the inventors employed the Phenograph K-nearest-neighbor density-based clustering algorithm. This unsupervised clustering analysis was performed on the data from single cells (25). To visualize the continuum of phenotypic cell populations, the output was organized using the Cluster Explorer plug-in, which generated an interactive cluster profile graph and heatmap, and displayed the cluster populations on a t-SNE plot.

Two-dimensional murine echocardiography. Echocardiography was performed using a Vevo 2100 high-resolution ultrasound system (VisualSonics, Toronto, ON, Canada) to measure changes in cardiac and physiology over time in a non-invasive manner, with the protocol adapted from Herrera et al. (2018) (26). A total of 9 mice (n=3 for normal pregnancy, n=3 for ascending infection, n=3 for nonpregnant control) were used for the study, evaluated on days 7, 14, 21, 28 and 60 postpartum. On the day of evaluation, mice were anesthetized with 1-2% isoflurane in oxygen delivered through a nosecone using a controlled-delivery anesthetic machine and were placed supine on a warmed platform with integrated electrocardiogramonitoring of heart rate, core temperature, and respiration. A sterile ophthalmic ointment (Artificial Tears, Akorn, Lake Forest, IL) was applied to the eyes to prevent them from drying out. Animal temperature was monitored with a rectal temperature probe, while pulse rate and respiration rate were monitored via electrode gel-lubricated electrode pads on the platform. Chest and abdominal hair were removed from the mice with a chemical hair removal lotion (Nair, Church & Dwight, Ewing, NJ). A 30-MHz transducer lubricated with high-viscosity ultrasound gel was used to evaluate the following parameters of cardiac function: heart rate (beats/min), cardiac output (mL/min) and ejection fraction (%). From the parasternal long axis (PLAX) view, the left ventricular outflow diameter and the blood flow through the aorta per unit time were measured. Cardiac output was calculated as the product of cross-sectional area×velocity time integral×heart rate. Systolic function was assessed from the parasternal short axis (PSAX) view by measuring end diastolic diameter (EDD) and end systolic diameter (ESD) using M-mode. Left ventricular ejection fraction was calculated as (EDD³−ESD³)/EDD³. Scanning took approximately 30 minutes per animal. Data was analyzed via one-way analysis of variance (ANOVA) with post-hoc Tukey's honestly significant difference (HSD) test using GraphPad Prism 9.5.0.

Fetal microchimeric cells migrate up to the maternal heart.

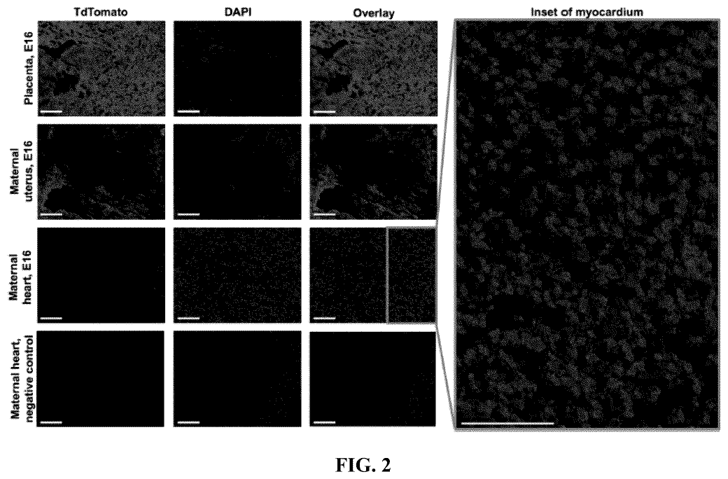

Immunofluorescence microscopy was employed to examine the presence of fetal microchimeric cells at the maternal-fetal interface and assess their distribution in the maternal heart. Tissues collected on E16 were subjected to this analysis, with the aim of identifying mT+ cells (FIG. 2). As anticipated, the placenta derived from the fetus displayed widespread expression of mT+. In the maternal uterus, mT+ cells were also observed, albeit mT+-expressing cells were detected in the maternal hearts on E16, although their fluorescence intensity was minimal (as depicted in the inset of FIG. 2). mT+ cells were also found in other maternal organs such as lungs and kidneys, but interestingly not in maternal brain (not shown). To rule out the possibility of autofluorescence, a negative control (maternal heart at E16 from wild-type female C57BL/6J mated with wild-type male C57BL/6J) which exhibited no red fluorescence.

Cell subpopulations in maternal hearts during normal pregnancy and during ascending infection.

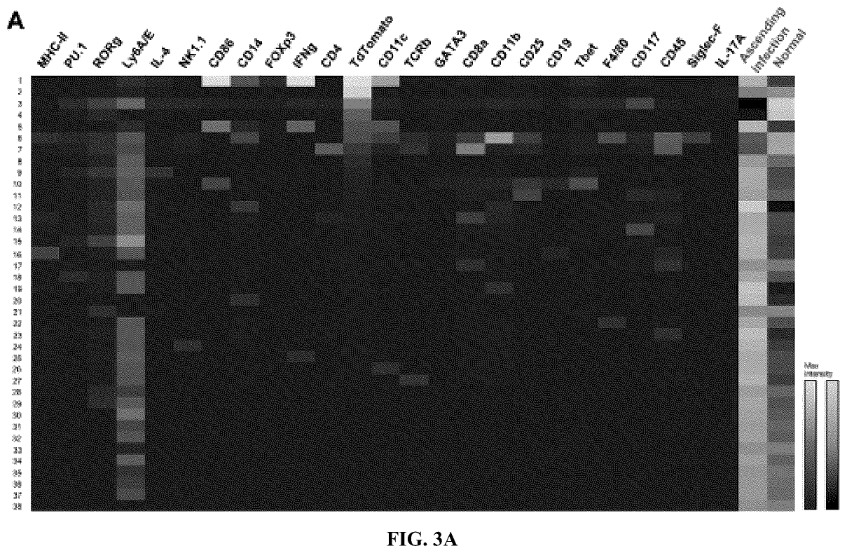

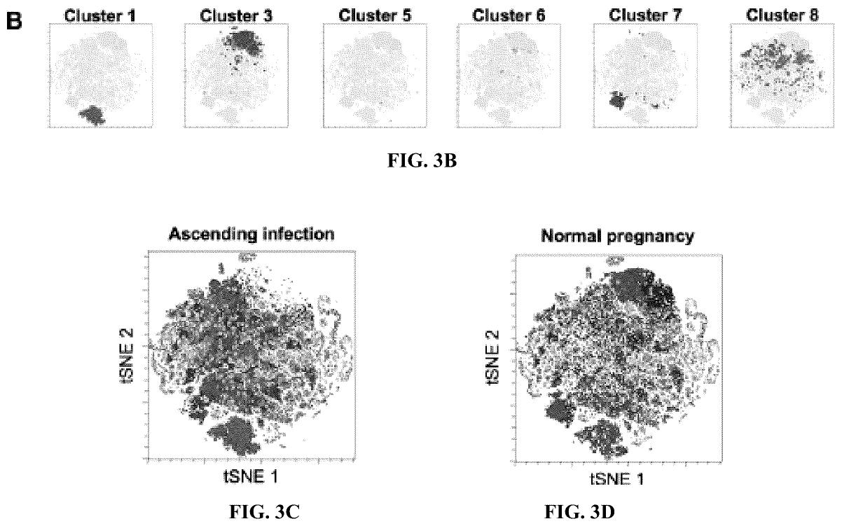

Among the 38 clusters (k=183) generated using Phenograph (FIG. 3A), 8 clusters were found to express mT+, representing approximately 28.76% of the cell population isolated from maternal heart. This frequency was most likely an overestimation of the fetal microchimeric population (usual population is <1 in 10,000 cells (27)) given that the single cell preparation was prepared with 70 μm filter, which would deplete most 100 μm-long rod-shaped maternally-derived cardiomyocytes (28). Cluster 3 exhibited the highest frequency among the mT+-positive cells at 7.85%, followed by cluster 1 at 6.77%, and cluster 8 at 5.78%. A summary of the identifiable mT+-expressing clusters based on high-dimensional mass cytometry data is presented in Table 1 and visualized in FIG. 3B. Cluster 1 expressed high CD14, CD11c, CD86 and IFN-γ which is indicative of a subpopulation of CD11c⁺ M1 macrophages. Cluster 3 expressed CD117 (c-kit) and Ly6A/E (Sca-1) and showed negative expression for lineage markers CD4, CD8, and CD11b (Mac-1), consistent with the Lineage-Sca-1⁺c-kit⁺ (LSK⁺) phenotype of murine hematopoietic cells. Cluster 5 was similar to Cluster 1, but with lower expression of CD14, pointing to an activated conventional dendritic cell phenotype. Cluster 6 expressed the monocyte-macrophage marker CD14, the myeloid marker CD11b, and F4/80, while being negative for macrophage polarization markers, indicating a non-activated macrophage phenotype. Cluster 7 expressed both CD4 and CD8, and low T cell receptor, characteristic of double-positive thymocytes. Cluster 8 was similar to cluster 3 except the former lacked expression of CD117 (c-kit), pointing to a Lineage⁻Sca-1⁺c-kit⁻ (LSK⁻) phenotype of murine hematopoietic cells called very small embryonic-like (VSEL) cells. Interestingly, clusters 2 and 4 did not exhibit expression of CD45 or other immune markers, suggesting a non-hematopoietic origin for these clusters. Among the identifiable clusters in Table 1, clusters 1, 5 and 8 showed an increase in frequency in ascending infection, whereas clusters 3, 6, and 7 displayed a decrease in frequency (FIGS. 3C and 3D).

TABLE 1
mT+ cell clusters in the maternal heart at E16.

| | | | Changes in |
|---|---|---|---|
| | | | pathologic |
| | | | vs normal |
| Cluster | Phenotype | Markers | pregnancy |
| 1 | CD11c⁺ M1 macrophages | CD14^{high}, CD11c⁺, | ↑↑ |
| | | CD86⁺, IFN-γ^{high} | |
| 3 | LSK⁺ hematopoietic stem | CD117⁺, Ly6A/E⁺, | ↓↓ |
| | cells | CD4⁻, CD8⁻, CD11b⁻ | |
| 5 | Activated conventional | CD14^{low}, CD11c⁺, | ↑↑ |
| | dendritic cells | CD86⁺, IFN-γ^{high} | |
| 6 | M0 (non-activated) | CD45⁺, CD14⁺, | ↓ |
| | macrophages | CD11b⁺, F4/80⁺ | |
| 7 | Double-positive thymocytes | CD45⁺, TCRb^{low}, | ↓ |
| | | CD4⁺, CD8⁺ | |
| 8 | Very small embryonic-like | CD117, Ly6A/E⁺, | ↑ |
| | (VSEL) cells | CD4⁻, CD8⁻, CD11b⁻ | |

Fetal microchimeric cells persist in postpartum maternal hearts.

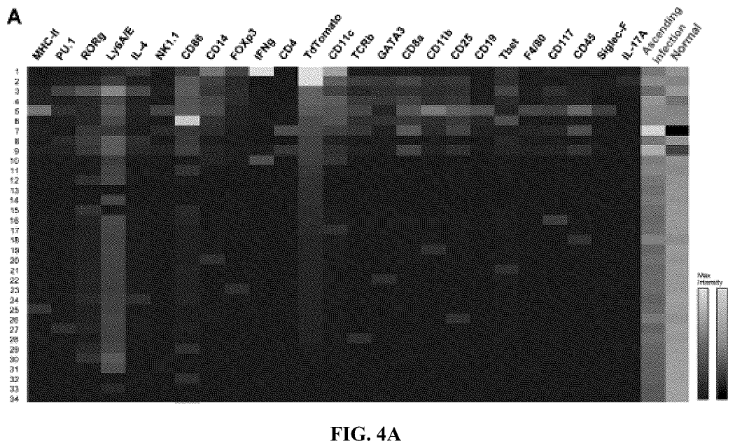

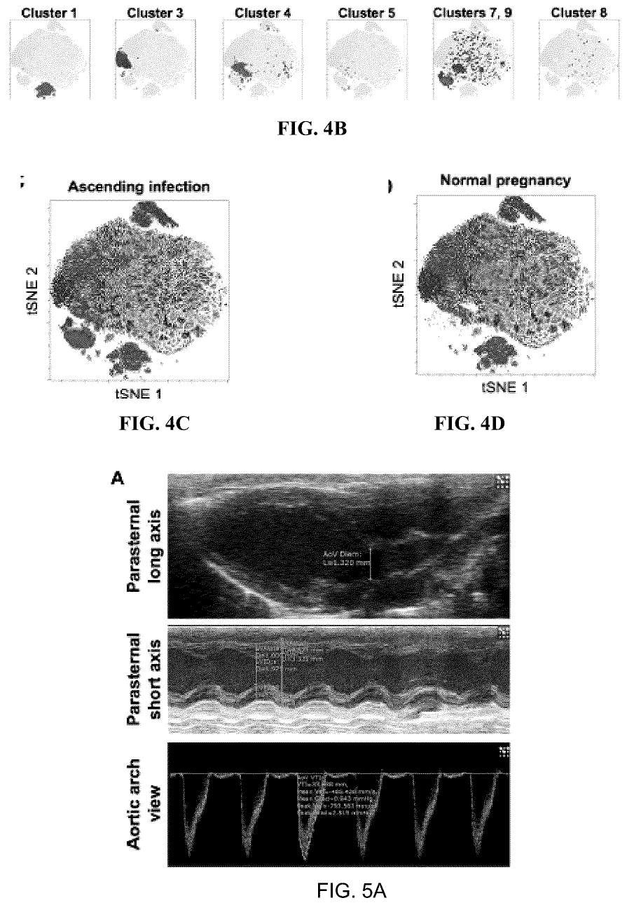

Thirty-four clusters were generated using Phenograph, 10 of which expressed TdTomato (FIG. 4A). Table 2 shows the identifiable mT+-expressing cell clusters, while FIG. 4B shows the cluster profiles in both ascending infection and normal pregnancy. Similar to E16, CD11c⁺ M1 macrophages (Cluster 1), LSK⁺ hematopoietic stem cells (Cluster 3), activated conventional dendritic cells (Cluster 4), double-positive thymocytes (Clusters 7 and 9) and very small embryonic-like (VSEL) cells (Cluster 8) were identified based on expression of similar markers. Interestingly, Cluster 5 expressed CD19, MHC-II, CD11b and CD25 which are cell markers for CD25⁺ B cells. Clusters 1, 3 and 8 showed a decrease in frequency with ascending infection, while clusters 4, 5, 7 and 9 showed an increase, with the latter two exhibiting marked increase in frequency (FIGS. 4C and 4D).

TABLE 2
mT+-expressing cell clusters in the maternal heart at PP28.

| | | | Changes in |
|---|---|---|---|
| | | | pathologic |
| | | | vs normal |
| Cluster | Phenotype | Markers | pregnancy |
| 1 | CD11c⁺ M1 macrophages | CD14^{high}, CD11c⁺, | ↓ |
| | | CD86⁺, IFN-γ^{high} | |
| 3 | LSK⁺ hematopoietic stem | CD117⁺, Ly6A/E⁺, | ↓ |
| | cells | CD4⁻, CD8⁻, CD11b⁻ | |
| 4 | Activated conventional | CD14^{low}, CD11c⁺, | ↑ |
| | dendritic cells | CD86⁺, IFN-γ^{high} | |
| 5 | CD25⁺ B cells | CD45⁺, CD19⁺, | ↑ |
| | | MHCII⁺, CD25⁺, | |
| | | CD11b⁺ | |
| 7 | Double-positive thymocytes | CD45⁺, TCRb^{low}, | ↑↑ |
| | | CD4⁺, CD8⁺ | |
| 8 | Very small embryonic-like | CD117⁻, Ly6A/E⁺, | ↓ |
| | (VSEL) cells | CD4⁻, CD8⁻, CD11b⁻ | |
| 9 | Double-positive thymocytes | CD45⁺, CD4⁺, CD8⁺ | ↑↑ |

Effect of ascending infection-induced preterm birth on postpartum maternal cardiovascular function.

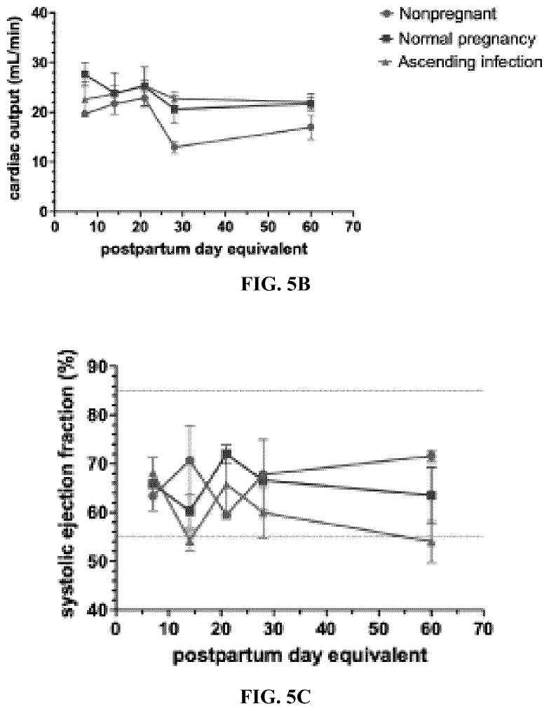

Baseline postpartum characteristics of the two pregnant mice groups. Of the 8 pregnant mice treated intravaginally with E. coli, 5 delivered live babies preterm, consistent with Spencer et al. (23). FIG. 5A shows various cardiac parameters measured via echocardiography up to 60 days postpartum. As shown in FIG. 5B, there was no difference in overall postpartum cardiac output between normal pregnancy and ascending infection groups, with cardiac output decreasing to pre-pregnancy levels before postpartum day 60.

Studying fetal microchimerism in humans and mice initially relied on the detection of SRY gene or Y chromosome present in male microchimeric cells (5, 6, 43), which underestimates female microchimeric cells (44-46). With the development of Cre reporter mouse models, fetal microchimeric cells and extracellular vesicles can be easily detected via expression of a fluorescent protein, differentiating them from maternally-derived cells and tissues (8, 21).

This study was able to show the following: (1) fetal microchimeric cells can escape the maternal-fetal interface and migrate to other maternal organs, including maternal heart; (2) there were differences in fetal microchimeric cell populations, particularly fetal antigen-presenting cells (APCs), fetal hematopoietic stem cells and fetal double-positive thymocytes, in normal pregnancy and ascending infection; (3) fetal microchimeric immune cells can persist up to 28 days postpartum, with the persistent cell populations different between normal pregnancy and ascending infection; and (4) no significant changes in maternal cardiac physiology were observed between normal pregnancy and ascending infection groups 60 days postpartum. The trend to reduce ejection fraction that was observed in all animals with pathologic pregnancies is indicative of the development of peripartum cardiomyopathy (42).

The primary purpose of the study is to characterize the cells in physiologic and pathologic pregnancies. One of the primary fetal microchimeric cell populations found in maternal hearts during pregnancy and postpartum was hematopoietic stem cells. The inventors found two fetal microchimeric bone marrow-derived stem cell populations: LSK⁺ and LSK⁻ cells. While LSK⁺ cells comprise the multipotent hematopoietic cells found in the bone marrow (32), LSK⁻ cells do not have long-term repopulation capacity or myeloid potential (38). Instead, these cells, also known as very small embryonice-like (VSEL) cells, are a heterogeneous cell population that contain early lymphoid-committed precursors distinct from common lymphoid progenitors (38). VSEL cells do not express Oct4A and are found to have higher apoptotic rate compared to LSK⁺ cells, which could be important in the regulation of survival of hematopoietic and leukemic stem cells (39, 58). In the high-dimensional single cell analysis, fetal microchimeric LSK⁺ and LSK⁻ hematopoietic stem cells did not persist in maternal hearts in ascending infection compared to normal pregnancy.

Another fetal microchimeric cell population detected in this analysis was double-positive thymocytes. These immature thymocytes, which developed from double-negative thymocytes, express both CD4 and CD8 along with TCR, which comprise three-quarters of all thymocytes (59, 60). These thymocytes then undergo positive selection wherein they escape apoptosis to become either CD4⁺ or CD8⁺ single-positive T cells (61). In response to bacterial or viral infection or stress, double-positive cells usually undergo apoptosis resulting in decreased thymic cellularity and atrophy (62). However, during pregnancy, escape of double-positive fetal thymocytes into the maternal periphery was observed instead, eventually settling down in maternal heart.

This phenomenon of thymocyte escape has been observed more in parasitic infections such as Trypanosoma cruzi (63, 64), Plasmodium berghei (65) or Schistosoma mansoni (66) than in bacterial or viral infections. Interestingly, decreased frequency of double-positive fetal thymocytes in maternal heart was initially observed with ascending infection. These fetal thymocytes, however, were cleared more following normal pregnancy, resulting in their persistence following an ascending infection.

Other fetal microchimeric immune cells detected in maternal heart were macrophages and dendritic cells. Both cell types form the mononuclear phagocyte system that is important in maintaining homeostasis in the cardiac tissue (68). These cells respond in response to ischemic injury and inflammation, ultimately resulting in fibrosis that can promote cardiac remodeling (69). By way of explanation, and in no way a limitation of the present invention, to limit formation of fibrotic scar, there must be less conventional dendritic cells and macrophages during the cardiac repair process.

In the postpartum data, higher fetal conventional dendritic cells and CD11c+ macrophages were seen in ascending infection, which may lead to cardiac remodeling especially in the presence of a stressor that can cause cardiac injury. These antigen-presenting cells, along with CD25+ B cells which were thought to be memory B cells (41), may present to maternal T cells via direct allorecognition (i.e., direct MHC presentation by fetal APCs) or semi-direct allorecognition (e.g., recycling and expression of fetal MHC by maternal APCs) (70).

With this reporter model, the inventors were able to detect fetal cells in not just in maternal heart but also in various maternal organs such as lungs and kidneys (data not shown), consistent with previous reports (1). The differences seen in immune cell subpopulations in maternal hearts depending on the context of pregnancy (physiologic vs. pathologic) demonstrate the selective trafficking of fetal microchimeric cells.

Interestingly, in a prospective cohort study looking into the impact of male-origin fetal microchimerism on cardiovascular risk, microchimerism was associated with decreased risk of ischemic heart disease (75), which supports that pregnancy can be beneficial to maternal cardiac health, provided that the pregnancy was not negatively impacted by disease etiologies such as preterm birth or preeclampsia.

In conclusion, different compositions of fetal microchimeric immune cells were found to migrate and persist in maternal heart, depending on the context of pregnancy (i.e., term or ascending infection-associated preterm delivery). These cells modulate the maternal cardiac tissue microenvironment.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

A risk score of the present disclosure may be calculated with an algorithm using well-known statistical analysis techniques. Non-limiting examples of statistical analysis techniques that may be used to calculate the risk score include cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms. In preferred embodiments, the risk score may be calculated using a random forest algorithm using the concentrations of three or more sample analytes in the panel of biomarkers. In an exemplary embodiment, the risk score is calculated as described in the examples.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. O'Donoghue K. Fetal microchimerism and maternal health during and after pregnancy. Obstet Med. 2008; 1(2):56-64.
2. Bianchi D W, Khosrotehrani K, Way S S, Mackenzie T C, Bajema I, O'Donoghue K. Forever Connected: The Lifelong Biological Consequences of Fetomaternal and Maternofetal Microchimerism. Clin Chem. 2021; 67(2):351-62.
3. Bianchi D W, Zickwolf G K, Weil G J, Sylvester S, DeMaria M A. Male fetal progenitor cells persist in maternal blood for as long as 27 years postpartum. Proc Natl Acad Sci USA. 1996; 93(2):705-8.
4. Chan W F, Nelson J L. Microchimerism in the human brain: more questions than answers. Chimerism. 2013; 4(1):32-3.
5. Bonney E A, Matzinger P. The maternal immune system's interaction with circulating fetal cells. J Immunol. 1997; 158(1):40-7.
6. Bonney E A, Onyekwuluje J. The H-Y response in mid-gestation and long after delivery in mice primed before pregnancy. Immunol Invest. 2003; 32(1-2):71-81.
7. Mahmood U, O'Donoghue K. Microchimeric fetal cells play a role in maternal wound healing after pregnancy. Chimerism. 2014; 5(2):40-52.
8. Kara R J, Bolli P, Karakikes I, Matsunaga I, Tripodi J, Tanweer O, et al. Fetal cells traffic to injured maternal myocardium and undergo cardiac differentiation. Circ Res. 2012; 110(1):82-93.
9. Sawaya H H, Jimenez S A, Artlett C M. Quantification of fetal microchimeric cells in clinically affected and unaffected skin of patients with systemic sclerosis. Rheumatology (Oxford). 2004; 43(8):965-8.
10. Kekow M, Barleben M, Drynda S, Jakubiczka S, Kekow J, Brune T. Long-term persistence and effects of fetal microchimerisms on disease onset and status in a cohort of women with rheumatoid arthritis and systemic lupus erythematosus. BMC Musculoskelet Disord. 2013; 14:325.
11. Johnson K L, McAlindon T E, Mulcahy E, Bianchi D W. Microchimerism in a female patient with systemic lupus erythematosus. Arthritis Rheum. 2001; 44(9):2107-11.
12. Blencowe H, Cousens S, Oestergaard M Z, Chou D, Moller A B, Narwal R, et al. National, regional, and worldwide estimates of preterm birth rates in the year 2010 with time trends since 1990 for selected countries: a systematic analysis and implications. Lancet. 2012; 379(9832):2162-72.
13. Cao G, Liu J, Liu M. Global, Regional, and National Incidence and Mortality of Neonatal Preterm Birth, 1990-2019. JAMA Pediatr. 2022; 176(8):787-96.
14. Hee Chung E, Chou J, Brown K A. Neurodevelopmental outcomes of preterm infants: a recent literature review. Transl Pediatr. 2020; 9 (Suppl 1):S3-S8.
15. Luu T M, Katz S L, Leeson P, Thebaud B, Nuyt A M. Preterm birth: risk factor for early-onset chronic diseases. CMAJ. 2016; 188(10):736-46.
16. Reddy U M, Rice M M, Grobman W A, Bailit J L, Wapner R J, Varner M W, et al. Serious maternal complications after early preterm delivery (24-33 weeks' gestation). Am J Obstet Gynecol. 2015; 213(4):538.e1-9.
17. Leahy-Warren P, Coleman C, Bradley R, Mulcahy H. The experiences of mothers with preterm infants within the first-year post discharge from NICU: social support, attachment and level of depressive symptoms. BMC Pregnancy Childbirth. 2020; 20(1):260.
18. Tanz L J, Stuart J J, Williams P L, Missmer S A, Rimm E B, James-Todd T M, et al. Preterm Delivery and Maternal Cardiovascular Disease Risk Factors: The Nurses' Health Study II. J Womens Health (Larchmt). 2019; 28(5):677-85.
19. Crump C, Sundquist J, Howell E A, McLaughlin M A, Stroustrup A, Sundquist K. Pre-Term Delivery and Risk of Ischemic Heart Disease in Women. J Am Coll Cardiol. 2020; 76(1):57-67.
20. Catov J M, Snyder G G, Fraser A, Lewis C E, Liu K, Althouse A D, et al. Blood Pressure Patterns and Subsequent Coronary Artery Calcification in Women Who Delivered Preterm Births. Hypertension. 2018; 72(1):159-66.
21. Sheller-Miller S, Choi K, Choi C, Menon R. Cyclic-recombinase-reporter mouse model to determine exosome communication and function during pregnancy. Am J Obstet Gynecol. 2019; 221(5):502.e1-e12.
22. Muzumdar M D, Tasic B, Miyamichi K, Li L, Luo L. A global double-fluorescent Cre reporter mouse. Genesis. 2007; 45(9):593-605.
23. Spencer N R, Radnaa E, Baljinnyam T, Kechichian T, Tantengco OAG, Bonney E, et al. Development of a mouse model of ascending infection and preterm birth. PLOS One. 2021; 16(12):e0260370.
24. Finck R, Simonds E F, Jager A, Krishnaswamy S, Sachs K, Fantl W, et al. Normalization of mass cytometry data with bead standards. Cytometry A. 2013; 83(5):483-94.
25. Levine J H, Simonds E F, Bendall S C, Davis K L, Amir e-A, Tadmor M D, et al. Data-Driven Phenotypic Dissection of AML Reveals Progenitor-like Cells that Correlate with Prognosis. Cell. 2015; 162(1):184-97.
26. Herrera S R, Vincent K L, Poole A, Olson G, Patrikeev I, Saada J, et al. Long-Term Effect of Lactation on Maternal Cardiovascular Function and Adiposity in a Murine Model. Am J Perinatol. 2019; 36(5):490-7.
27. Fjeldstad H E, Johnsen G M, Staff A C. Fetal microchimerism and implications for maternal health. Obstet Med. 2020; 13(3):112-9.
28. Miranda A M A, Janbandhu V, Maatz H, Kanemaru K, Cranley J, Teichmann S A, et al. Single-cell transcriptomics for the assessment of cardiac disease. Nat Rev Cardiol. 2023; 20(5):289-308.
29. Zhou Y, Yoshida S, Kubo Y, Yoshimura T, Kobayashi Y, Nakama T, et al. Different distributions of M1 and M2 macrophages in a mouse model of laser-induced choroidal neovascularization. Mol Med Rep. 2017; 15(6):3949-56.
30. Motomura Y, Kanno S, Asano K, Tanaka M, Hasegawa Y, Katagiri H, et al. Identification of Pathogenic Cardiac CD11c+ Macrophages in Nodl-Mediated Acute Coronary Arteritis. Arterioscler Thromb Vasc Biol. 2015; 35(6):1423-33.
31. Munder M, Mallo M, Eichmann K, Modolell M. Murine macrophages secrete interferon gamma upon combined stimulation with interleukin (IL)-12 and IL-18: A novel pathway of autocrine macrophage activation. J Exp Med. 1998; 187(12):2103-8.
32. Challen G A, Boles N, Lin K K, Goodell M A. Mouse hematopoietic stem cell identification and analysis. Cytometry A. 2009; 75(1): 14-24.
33. Kim M K, Kim J. Properties of immature and mature dendritic cells: phenotype, morphology, phagocytosis, and migration. RSC Adv. 2019; 9(20):11230-8.
34. Vremec D, O'Keeffe M, Hochrein H, Fuchsberger M, Caminschi I, Lahoud M, et al. Production of interferons by dendritic cells, plasmacytoid cells, natural killer cells, and interferon-producing killer dendritic cells. Blood. 2007; 109(3):1165-73.
35. Dos Anjos Cassado A. F4/80 as a Major Macrophage Marker: The Case of the Peritoneum and Spleen. Results Probl Cell Differ. 2017; 62:161-79.
36. Li Y, Li K, Zhu L, Li B, Zong D, Cai P, et al. Development of double-positive thymocytes at single-cell resolution. Genome Med. 2021; 13(1):49.
37. Brodeur J F, Li S, Damlaj O, Dave V P. Expression of fully assembled TCR-CD3 complex on double positive thymocytes: synergistic role for the PRS and E R retention motifs in the intra-cytoplasmic tail of CD3epsilon. Int Immunol. 2009; 21(12):1317-27.
38. Kumar R, Fossati V, Israel M, Snoeck H W. Lin-Scal+kit− bone marrow cells contain early lymphoid-committed precursors that are distinct from common lymphoid progenitors. J Immunol. 2008; 181(11):7507-13.
39. Szade K, Bukowska-Strakova K, Nowak W N, Szade A, Kachamakova-Trojanowska N, Zukowska M, et al. Murine bone marrow Lin⁻Sca⁻1⁺CD45⁻ very small embryonic-like (VSEL) cells are heterogeneous population lacking Oct-4A expression. PLOS One. 2013; 8(5):e63329.
40. Ma J K, Platt M Y, Eastham-Anderson J, Shin J S, Mellman I. MHC class II distribution in dendritic cells and B cells is determined by ubiquitin chain length. Proc Natl Acad Sci USA. 2012; 109(23):8820-7.
41. Amu S, Gjertsson I, Brisslert M. Functional characterization of murine CD25 expressing B cells. Scand J Immunol. 2010; 71(4):275-82.
42. Bhattacharyya A, Basra S S, Sen P, Kar B. Peripartum cardiomyopathy: a review. Tex Heart Inst J. 2012; 39(1):8-16.
43. Bonney E A, Onyekwuluje J. Maternal tolerance to H-Y is independent of IL-10. Immunol Invest. 2004; 33(4):385-95.
44. Yan Z, Lambert N C, Ostensen M, Adams K M, Guthrie K A, Nelson J L. Prospective study of fetal DNA in serum and disease activity during pregnancy in women with inflammatory arthritis. Arthritis Rheum. 2006; 54(7):2069-73.
45. Nemescu D, Ursu R G, Nemescu E R, Negura L. Heterogeneous Distribution of Fetal Microchimerism in Local Breast Cancer Environment. PLOS One. 2016; 11(1):e0147675.
46. Tan X W, Liao H, Sun L, Okabe M, Xiao Z C, Dawe G S. Fetal microchimerism in the maternal mouse brain: a novel population of fetal progenitor or stem cells able to cross the blood-brain barrier? Stem Cells. 2005; 23(10):1443-52.
47. Chen S L, Fang W W, Ye F, Liu Y H, Qian J, Shan S J, et al. Effect on left ventricular function of intracoronary transplantation of autologous bone marrow mesenchymal stem cell in patients with acute myocardial infarction. Am J Cardiol. 2004; 94(1):92-5.
48. Wollert K C, Meyer G P, Lotz J, Ringes-Lichtenberg S, Lippolt P, Breidenbach C, et al. Intracoronary autologous bone-marrow cell transfer after myocardial infarction: the BOOST randomised controlled clinical trial. Lancet. 2004; 364(9429): 141-8.
49. Janssens S, Dubois C, Bogaert J, Theunissen K, Deroose C, Desmet W, et al. Autologous bone marrow-derived stem-cell transfer in patients with ST-segment elevation myocardial infarction: double-blind, randomised controlled trial. Lancet. 2006; 367(9505): 113-21.
50. Lunde K, Solheim S, Aakhus S, Arnesen H, Abdelnoor M, Forfang K, et al. Autologous stem cell transplantation in acute myocardial infarction: The ASTAMI randomized controlled trial. Intracoronary transplantation of autologous mononuclear bone marrow cells, study design and safety aspects. Scand Cardiovasc J. 2005; 39(3):150-8.
51. Choudry F, Hamshere S, Saunders N, Veerapen J, Bavnbek K, Knight C, et al. A randomized double-blind control study of early intra-coronary autologous bone marrow cell infusion in acute myocardial infarction: the REGENERATE-AMI clinical trialt. Eur Heart J. 2016; 37(3):256-63.
52. Jackson K A, Majka S M, Wang H, Pocius J, Hartley C J, Majesky M W, et al. Regeneration of ischemic cardiac muscle and vascular endothelium by adult stem cells. J Clin Invest. 2001; 107(11):1395-402.
53. Orlic D, Kajstura J, Chimenti S, Jakoniuk I, Anderson S M, Li B, et al. Bone marrow cells regenerate infarcted myocardium. Nature. 2001; 410(6829):701-5.
54. Murry C E, Soonpaa M H, Reinecke H, Nakajima H, Nakajima H O, Rubart M, et al. Haematopoietic stem cells do not transdifferentiate into cardiac myocytes in myocardial infarcts. Nature. 2004; 428(6983):664-8.
55. Nygren J M, Jovinge S, Breitbach M, Säwen P, Roll W, Hescheler J, et al. Bone marrow-derived hematopoietic cells generate cardiomyocytes at a low frequency through cell fusion, but not transdifferentiation. Nat Med. 2004; 10(5):494-501.
56. Alvarez-Dolado M, Pardal R, Garcia-Verdugo J M, Fike J R, Lee H O, Pfeffer K, et al. Fusion of bone-marrow-derived cells with Purkinje neurons, cardiomyocytes and hepatocytes. Nature. 2003; 425(6961):968-73.
57. Khosrotehrani K, Leduc M, Bachy V, Nguyen Huu S, Oster M, Abbas A, et al. Pregnancy allows the transfer and differentiation of fetal lymphoid progenitors into functional T and B cells in mothers. J Immunol. 2008; 180(2):889-97.
58. Peng C, Chen Y, Shan Y, Zhang H, Guo Z, Li D, et al. LSK derived LSK− cells have a high apoptotic rate related to survival regulation of hematopoietic and leukemic stem cells. PLOS One. 2012; 7(6):e38614.
59. de Meis J, Aurélio Farias-de-Oliveira D, Nunes Panzenhagen P H, Maran N, Villa-Verde D M, Morrot A, et al. Thymus atrophy and double-positive escape are common features in infectious diseases. J Parasitol Res. 2012; 2012:574020.
60. Matsumoto K, Yoshikai Y, Moroi Y, Asano T, Ando T, Nomoto K. Two differential pathways from double-negative to double-positive thymocytes. Immunology. 1991; 72(1):20-6.
61. Savino W, Dardenne M, Velloso L A, Dayse Silva-Barbosa S. The thymus is a common target in malnutrition and infection. Br J Nutr. 2007; 98 Suppl 1:S11-6.
62. Dooley J, Liston A. Molecular control over thymic involution: from cytokines and microRNA to aging and adipose tissue. Eur J Immunol. 2012; 42(5):1073-9.
63. Mendes-da-Cruz D A, Silva J S, Cotta-de-Almeida V, Savino W. Altered thymocyte migration during experimental acute Trypanosoma cruzi infection: combined role of fibronectin and the chemokines CXCL12 and CCL4. Eur J Immunol. 2006; 36(6): 1486-93.
64. Mendes-da-Cruz D A, de Meis J, Cotta-de-Almeida V, Savino W. Experimental Trypanosoma cruzi infection alters the shaping of the central and peripheral T-cell repertoire. Microbes Infect. 2003; 5(10):825-32.
65. Francelin C, Paulino L C, Gameiro J, Verinaud L. Effects of Plasmodium berghei on thymus: high levels of apoptosis and premature egress of CD4(+)CD8(+) thymocytes in experimentally infected mice. Immunobiology. 2011; 216(10): 1148-54.
66. Wellhausen S R, Boros D L. Atrophy of the thymic cortex in mice with granulomatous schistosomiasis mansoni. Infect Immun. 1982; 35(3):1063-9.
67. Morrot A, Terra-Granado E, Pérez A R, Silva-Barbosa S D, Milićević N M, Farias-de-Oliveira D A, et al. Chagasic thymic atrophy does not affect negative selection but results in the export of activated CD4+CD8+ T cells in severe forms of human disease. PLOS Negl Trop Dis. 2011; 5(8):e1268.
68. Van der Borght K, Lambrecht B N. Heart macrophages and dendritic cells in sickness and in health: A tale of a complicated marriage. Cell Immunol. 2018; 330:105-13.
69. Simões F C, Riley P R. Immune cells in cardiac repair and regeneration. Development. 2022; 149(8).
70. Murrieta-Coxca J M, Fuentes-Zacarias P, Ospina-Prieto S, Markert U R, Morales-Prieto D M. Synergies of Extracellular Vesicles and Microchimerism in Promoting Immunotolerance During Pregnancy. Front Immunol. 2022; 13:837281.
71. Seppanen E J, Hodgson S S, Khosrotehrani K, Bou-Gharios G, Fisk N M. Fetal microchimeric cells in a fetus-treats-its-mother paradigm do not contribute to dystrophin production in serially parous mdx females. Stem Cells Dev. 2012; 21(15):2809-16.
72. Kolialexi A, Tsangaris G T, Antsaklis A, Mavroua A. Rapid clearance of fetal cells from maternal circulation after delivery. Ann N Y Acad Sci. 2004; 1022:113-8.
73. Gammill H S, Nelson J L. Naturally acquired microchimerism. Int J Dev Biol. 2010; 54(2-3):531-43.
74. Pritchard S, Peter I, Johnson K L, Bianchi D W. The natural history of fetal cells in postpartum murine maternal lung and bone marrow: a two-stage phenomenon. Chimerism. 2012; 3(3):59-64.
75. Hallum S, Gerds T A, Sehested TSG, Jakobsen M A, Tjønneland A, Kamper-Jørgensen M. Impact of Male-Origin Microchimerism on Cardiovascular Disease in Women: A Prospective Cohort Study. Am J Epidemiol. 2021; 190(5):853-63.
76. Prell R A, Halpern W G, Rao G K. Perspective on a Modified Developmental and Reproductive Toxicity Testing Strategy for Cancer Immunotherapy. Int J Toxicol. 2016; 35(3):263-73.
77. Popli R, Sahaf B, Nakasone H, Lee J Y, Miklos D B. Clinical impact of H-Y alloimmunity. Immunol Res. 2014; 58(2-3):249-58.
78. Kawada H, Fujita J, Kinjo K, Matsuzaki Y, Tsuma M, Miyatake H, et al. Nonhematopoietic mesenchymal stem cells can be mobilized and differentiate into cardiomyocytes after myocardial infarction. Blood. 2004; 104(12):3581-7.
79. Fukuda K, Fujita J. Mesenchymal, but not hematopoietic, stem cells can be mobilized and differentiate into cardiomyocytes after myocardial infarction in mice. Kidney Int. 2005; 68(5):1940-3.
80. Wu L, Dalal R, Cao C D, Postoak J L, Yang G, Zhang Q, et al. IL-10-producing B cells are enriched in murine pericardial adipose tissues and ameliorate the outcome of acute myocardial infarction. Proc Natl Acad Sci USA. 2019; 116(43):21673-84.
81. Chen R, Liu F, Xia L, Che N, Tian Y, Cao Y, et al. B10 cells decrease fibrosis progression following cardiac injury partially by IL-10 production and regulating hyaluronan secretion. J Leukoc Biol. 2022; 111(2):415-25.
82. Krenek P, Kmecova J, Kucerova D, Bajuszova Z, Musil P, Gazova A, et al. Isoproterenol-induced heart failure in the rat is associated with nitric oxide-dependent functional alterations of cardiac function. Eur J Heart Fail. 2009; 11(2): 140-6.
83. Brooks W W, Conrad C H. Isoproterenol-induced myocardial injury and diastolic dysfunction in mice: structural and functional correlates. Comp Med. 2009; 59(4):339-43.

What is claimed is:

1. A method of determining an increased risk of maternal cardiovascular disease caused by an infection comprising:
obtaining, or having obtained, a biological sample from a subject at risk of, of having had a pre-term birth;
measuring fetal microchimeric cells or fetal extracellular vesicles in the biological sample; and
using a machine learning algorithm calculating a risk of cardiovascular disease based on an increase or a decrease in fetal microchimeric cells or fetal extracellular vesicles in the biological sample when compared to a maternal sample from a subject that does not have an infection.

2. The method of claim 1, wherein at least one of:
an increase in a frequency of fetal microchimeric cells or fetal extracellular vesicles expressed high CD14 or CD14^{low}, CD11c, CD86 and IFN-γ which is indicative of a subpopulation of CD11c+ M1 macrophages or dendritic cell phenotypes;
an increase in a frequency of fetal microchimeric cells or fetal extracellular vesicles expressed CD117 (c-kit) and Ly6A/E (Sca-1) and showed negative expression for lineage markers CD4, CD8, and CD11b (Mac-1), consistent with Lineage-Sca-1+c-kit+ (LSK+) phenotype of murine hematopoietic cells; or
an increase in a frequency of fetal microchimeric cells or fetal extracellular vesicles comprising Lineage-Sca-1+c-kit− (LSK−) phenotype of murine hematopoietic cells called very small embryonic-like (VSEL) cells.

3. The method of claim 1, wherein there is at least one of:
a decrease in a frequency of fetal microchimeric cells or fetal extracellular vesicles for monocyte-macrophage marker CD14, myeloid marker CD11b, and F4/80, while being negative for macrophage polarization markers, indicating a non-activated macrophage phenotype;
a decrease in a frequency of fetal microchimeric cells or fetal extracellular vesicles expressed both CD4 and CD8, and low T cell receptor, characteristic of double-positive thymocytes; or
a decrease in a frequency of fetal microchimeric cells or fetal extracellular vesicles of non-hematopoietic that do not express CD45.

4. The method of claim 1, wherein the subject also has an increase in a risk of hypertension, coronary artery calcification, type 2 diabetes mellitus and hypercholesterolemia.

5. The method of claim 1, wherein the biological sample is a blood, a plasma, cardiac, a kidney, or a lung tissue.

6. The method of claim 1, wherein based on the calculated risk of cardiovascular disease is an increase in a risk of cardiovascular disease as a result of an infection administering one or more anti-infective agent, antibiotic agent, or antimicrobial agent to the subject to treat the infection.

7. The method of claim 1, wherein the antibiotic is selected from the group consisting of: azithromycin penicillins, cephalosporines, tetracyclines, sulphonamides, aminoglycosides, aminocyclitols, macrolides, quinolones, ionophores, carbadox, nitrofuran antibiotics, phenicols, a mixture thereof, and any combination thereof.

8. The method of claim 1, wherein the antibiotic is selected from the group consisting of: a macrolide, aminoglycoside, polymyxin, a tetracycline, a cephalosporin, a quinolone or a fluoroquinolone; amikacin, apramycin, gentamicin, kanamycin, neomycin, tobramycin, paromomycin, streptomycin, spectinomycin, plazomicin, ertapenem, doripenem, imipenem/cilastatin, meropenem, cefadroxil, cefazolin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, clindamycin, lincomycin, lipopeptide, daptomycin, azithromycin, clarithromycin, dirithromycin, erythromycin, roxithromycin, troleandomycin, telithromycin, spiramycin, fidaxomicin, tulathromycin, aztreonam, linezolid, posizolid, radezolid, torezolid, amoxicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, bacitracin, colistin, polymyxin b, besifloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, sulfonamidochrysoidine, demeclocycline, doxycycline, minocycline, oxytetracycline, tetracycline, arsphenamine, chloramphenicol, fosfomycin, fusidic acid, metronidazole, mupirocin, platensimycin, quinupristin/dalfopristin, thiamphenicol, tigecycline, tinidazole, trimethoprim, clofazimine, dapsone, capreomycin, cycloserine, ethambutol, ethionamide, isoniazid, pyrazinamide, rifampicin, rifabutin, or rifapentine, or a pharmaceutically acceptable salt thereof.

9. The method of claim 1, wherein the machine learning algorithm is selected from perplexity, learning rate (eta), K-Nearest Neighbors algorithm (exact, vantage point tree), and gradient algorithm (Barnes-Hut) and phenograph K-nearest-neighbor density-based clustering algorithm.

10. A method of treating a subject that is pregnant and suspected of having an increased risk of maternal cardiovascular disease caused by an infection comprising:
obtaining, or having obtained, a biological sample from a subject at risk of, of having had a pre-term birth;
measuring fetal microchimeric cells or fetal extracellular vesicles in the biological sample; and
using a machine learning algorithm calculating a risk of cardiovascular disease based on an increase or a decrease in fetal microchimeric cells or fetal extracellular vesicles in the biological sample when compared to a maternal sample from a subject that does not have an infection; and
based on the calculated risk of cardiovascular disease administering one or more anti-infective agents, antibiotic agents, or antimicrobial agents to the subject.

11. The method of claim 10, wherein at least one of:
an increase in a frequency of fetal microchimeric cells or fetal extracellular vesicles expressed high CD14 or CD14^{low}, CD11c, CD86 and IFN-γ which is indicative of a subpopulation of CD11c+ M1 macrophages or dendritic cell phenotypes;
an increase in a frequency of fetal microchimeric cells or fetal extracellular vesicles expressed CD117 (c-kit) and Ly6A/E (Sca-1) and showed negative expression for lineage markers CD4, CD8, and CD11b (Mac-1), consistent with Lineage-Sca-1+c-kit+ (LSK+) phenotype of murine hematopoietic cells; or
an increase in a frequency of fetal microchimeric cells or fetal extracellular vesicles comprising Lineage-Sca-1+c-kit− (LSK−) phenotype of murine hematopoietic cells called very small embryonic-like (VSEL) cells.

12. The method of claim 10, wherein there is at least one of:
a decrease in a frequency of fetal microchimeric cells or fetal extracellular vesicles for monocyte-macrophage marker CD14, myeloid marker CD11b, and F4/80, while being negative for macrophage polarization markers, indicating a non-activated macrophage phenotype;
a decrease in a frequency of fetal microchimeric cells or fetal extracellular vesicles expressed both CD4 and CD8, and low T cell receptor, characteristic of double-positive thymocytes; or
a decrease in a frequency of fetal microchimeric cells or fetal extracellular vesicles of non-hematopoietic that do not express CD45.

13. The method of claim 10, wherein the subject also has an increase in a risk of hypertension, coronary artery calcification, type 2 diabetes mellitus and hypercholesterolemia.

14. The method of claim 10, wherein the biological sample is a blood, a plasma, cardiac, a kidney or a lung tissue.

15. The method of claim 10, wherein the antibiotic is selected from the group consisting of: azithromycin penicillins, cephalosporines, tetracyclines, sulphonamides, aminoglycosides, aminocyclitols, macrolides, quinolones, ionophores, carbadox, nitrofuran antibiotics, phenicols, a mixture thereof, and any combination thereof.

16. The method of claim 10, wherein the antibiotic is selected from the group consisting of: a macrolide, aminoglycoside, polymyxin, a tetracycline, a cephalosporin, a quinolone or a fluoroquinolone; amikacin, apramycin, gentamicin, kanamycin, neomycin, tobramycin, paromomycin, streptomycin, spectinomycin, plazomicin, ertapenem, doripenem, imipenem/cilastatin, meropenem, cefadroxil, cefazolin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, clindamycin, lincomycin, lipopeptide, daptomycin, azithromycin, clarithromycin, dirithromycin, erythromycin, roxithromycin, troleandomycin, telithromycin, spiramycin, fidaxomicin, tulathromycin, aztreonam, linezolid, posizolid, radezolid, torezolid, amoxicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, bacitracin, colistin, polymyxin b, besifloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, sulfonamidochrysoidine, demeclocycline, doxycycline, minocycline, oxytetracycline, tetracycline, arsphenamine, chloramphenicol, fosfomycin, fusidic acid, metronidazole, mupirocin, platensimycin, quinupristin/dalfopristin, thiamphenicol, tigecycline, tinidazole, trimethoprim, clofazimine, dapsone, capreomycin, cycloserine, ethambutol, ethionamide, isoniazid, pyrazinamide, rifampicin, rifabutin, or rifapentine, or a pharmaceutically acceptable salt thereof.

17. The method of claim 10, wherein the machine learning algorithm is selected from perplexity, learning rate (eta), K-Nearest Neighbors algorithm (exact, vantage point tree), and gradient algorithm (Barnes-Hut) and phenograph K-nearest-neighbor density-based clustering algorithm.

18. The method of claim 10, wherein a computerized method for determining an increased risk of maternal cardiovascular disease caused by an infection further comprises using a processor with a non-transitory computer readable medium and a machine learning algorithm to calculate a risk of cardiovascular disease based on an increase or a decrease in fetal microchimeric cells in the biological sample when compared to a maternal sample from a subject that does not have an infection.

19. A method of treating a subject in need of regenerating or protecting one or more maternal organs comprising:
isolating at least one of: fetal microchimeric cells or fetal extracellular vesicles comprising stem cell-like properties from a pregnant female during pregnancy; and
injecting the fetal microchimeric cells or fetal extracellular vesicles into a patient in need thereof in an amount sufficient to regenerate and protect the one or more maternal organs.

20. The method of claim 19, wherein the one or more organs are selected from heart, kidney, or lung.

21. The method of claim 19, wherein the cells are not obtained from a pregnant female subject with a premature birth or preterm pregnancy.

22. The method of claim 19, wherein the at least one of fetal microchimeric cells or fetal extracellular vesicles are selected from:
fetal microchimeric cells or fetal extracellular vesicles that express high CD14 or CD14^{low}, CD11c, CD86 and IFN-γ which is indicative of a subpopulation of CD11c+ M1 macrophages or dendritic cell phenotypes;
fetal microchimeric cells or fetal extracellular vesicles that express CD117 (c-kit) and Ly6A/E (Sca-1) and showed negative expression for lineage markers CD4, CD8, and CD11b (Mac-1), consistent with Lineage-Sca-1+c-kit+ (LSK+) phenotype of murine hematopoietic cells; or
fetal microchimeric cells or fetal extracellular vesicles that express comprising Lineage-Sca-1+c-kit− (LSK−) phenotype of murine hematopoietic cells called very small embryonic-like (VSEL) cells;
fetal microchimeric cells or fetal extracellular vesicles comprising monocyte-macrophage marker CD14, myeloid marker CD11b, and F4/80, while being negative for macrophage polarization markers, indicating a non-activated macrophage phenotype;
fetal microchimeric cells or fetal extracellular vesicles comprising that express both CD4 and CD8, and low T cell receptor, characteristic of double-positive thymocytes; or
fetal microchimeric cells or fetal extracellular vesicles of non-hematopoietic that do not express CD45.
